(19) United States
(12) Patent Application Publication
(10) Pub. No.: US 2002/0007135 A1
(43) Pub. Date: Jan. 17, 2002

(54) Device for shielding tissue

(76) Inventor: David L. Thomas III, Chicago, IL (US)

Correspondence Address: A. Blair Hughes, McDonnell Boehnen Hulbert & Berghoff, 32nd Floor, 300 S. Wacker Drive, Chicago, IL, 60606, US

(21) Appl. No.: 09/894,299

(22) Filed: Jun. 28, 2001

Related U.S. Application Data
(60) Provisional application No. 60/214,575, filed on Jun. 28, 2000.

(51) Int. Cl. A61F 13/00

(52) U.S. Cl. 602/41

(57) ABSTRACT

Shielding devices for protecting tissue from contaminants including a flexible membrane forming a tubular structure and having an internal surface, an external surface, and two ends.

### FIELD OF THE INVENTION

The present invention relates to a flexible and disposable shielding device for protecting skin from an external contaminant.

### BACKGROUND OF THE INVENTION

There is a need for an inexpensive and easy to use device that provides for the protection of skin surfaces from external elements such as water. Skin surface may be subject to a wound, an IV shunt, stitches, staples or other disturbances and injuries. Each type of disturbance or injury requires that the skin surface be protected from external elements on a periodic or continuous basis. For instance, hospital patients are often fitted with an IV shunt. Should the patient desire to shower or otherwise bathe, the patient is left with very few options. In fact, in order to provide the greatest amount of protection, the shunt is typically sealed using tape and a rubber glove or removed and replaced with a new one after the shower. Both options are painful and cause the patient discomfort. The removal of the tape can result in removal of hair and pain. The patient is also subjected to pain upon reinsertion of the IV shunt. Thus, a device that would allow a patient to painlessly protect the skin surface without the use of tape or removal of the IV shunt would be very useful.

Provided herein is such a device, as described below.

### BRIEF DESCRIPTION OF THE DRAWINGS

FIG. 1 is a view of a human arm including an embodiment of a tissue shielding device of this invention.

FIG. 2A ls a side view and FIG. 2B is a perspective view of an embodiment of a tissue shielding device of this invention.

### SUMMARY OF THE INVENTION

The present invention relates to shielding devices for protecting tissue from contaminants. The shielding device comprises a flexible membrane forming a tubular structure and having an internal surface, an external surface, and two ends. The external surface is preferably resistant to external elements such as water, dust, dirt and infectious agents such as bacteria. In certain embodiments, the device is impervious to a virus such as the Human Immunodeficiency Virus (HIV). The device also optionally includes one or more sealing mechanisms that apply pressure near one or both ends of the device, effectively sealing tissue covered by the device from external elements following application to an appendage, such as a forearm.

In one embodiment, this invention is a skin shielding device comprising a flexible membrane forming a cylinder having an internal surface, an external surface, a first end, and a second end wherein the external surface is impermeable to at least one contaminant, said membrane including at least one first seal.

In another embodiment, this invention is a method for applying a skin shielding device to an appendage. The shielding device used comprises a flexible membrane forming a cylinder having an internal surface, an external surface, a first end, and a second end wherein the external surface is impermeable to at least one contaminant, said membrane including at least one first seal. The sealing device is applied to an appendage by the steps comprising placing an end of the appendage into the first end of the skin shielding device; and passing the end of the appendage through the skin shielding device until a portion of the appendage passes out of the second end of the skin shielding device. The sealing device is useful for protecting an appendage is selected from the group consisting of an arm, a leg, a hand, a foot, a finger, and toe, a torso, and a head from contaminants. The method is especially useful for protecting appendages including an IV shunt or a wound from contaminants or from getting wet during bathing.

### DETAILED DESCRIPTION OF THE INVENTION

Provided herein is a shielding device for protecting a mammalian limb or appendage such as a forearm, hand, or foot from contacting contaminants such as dirt, moisture, chemicals, viruses, bacteria and other, similar contaminants. The shielding device may be used to protect the limb or appendage of a human being or other mammal, such as a dog, horse, or cow. The shielding devices are especially useful for shielding a wound or other skin defect such as a defect that may occur by the insertion of an IV shunt, stitches, or staples, for example, from contaminants.

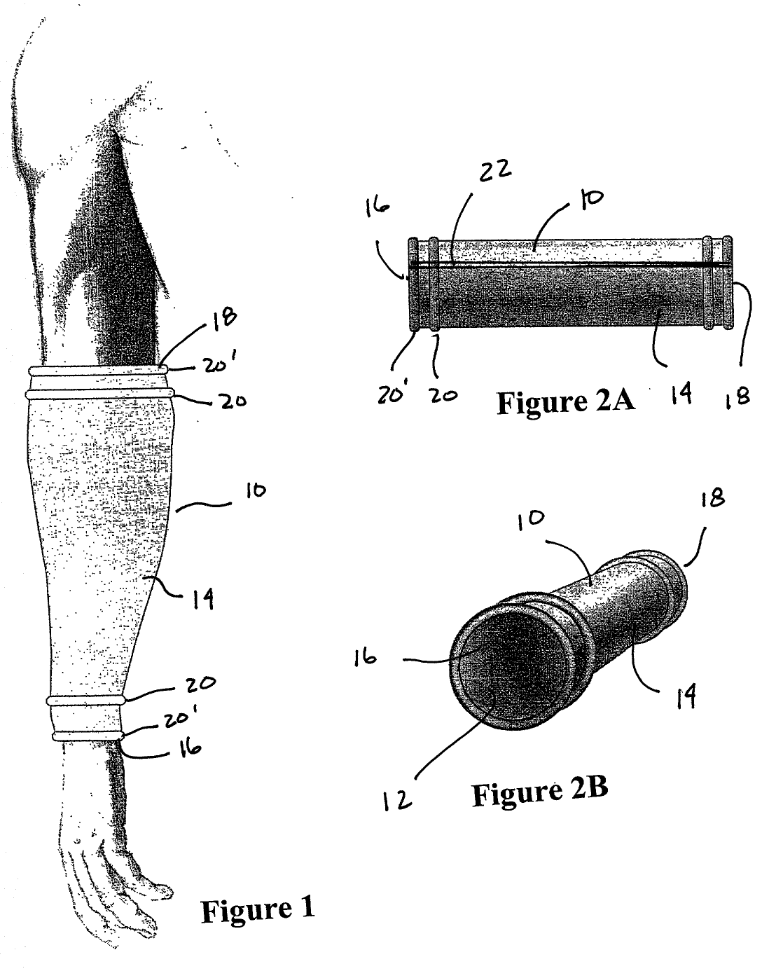

Referring now to FIGS. 1, 2A and 2B, a shielding device 10 of this invention is constructed as a flexible membrane in the form of a tubular structure that includes an internal surface 12 and an external surface 14 that define first end 16 and a second end 18. First end 16 and second end 18 may have the same or different cross-section areas. In addition, first end 16 and second end 18 may have a circular or non-circular cross-section with a circular cross-section being preferred.

External surface 14 of shielding device 10 is resistant to at least one contaminant and is preferably resistant to most or all contaminants such as water, dust, dirt and infectious agents such as bacteria. In certain embodiments, sealing device 10 is impervious to a virus such as the Human Immunodeficiency Virus (HIV). It is preferred that shielding device 10 is air permeable.

In an embodiment of this invention, sealing device 10 is constructed of a sufficiently breathable rubber substance that allows for the exchange of air but not other elements between internal surface 12 and the external surface 14 of sealing device 10. Thus, in one embodiment, sealing device 10 is manufactured of a membrane material that is sufficiently non-porous so as to prevent the passage and accumulation of contaminants near the skin. In order to be essentially impervious to contaminants and/or flexible, shielding device 10 will typically be manufactured from a flexible membranous material such a rubber, synthetic rubber, latex or any other material that is flexible and compatible with skin or tissue.

Shielding device 10 may include a first seal 20 at one or both ends 16 and 18 of shielding device 10. One purpose of first seal 20 is to urge an end of sealing device 10 into contact with the skin or body part being protected, effectively closing the end of the shielding device 10 thereby inhibiting the passage of contaminants through the closed end and into contact with the portion of the body covered by shielding device 10.

First seal 20 may be any type of seal that is capable of urging a flexible cylinder into contact with a concentric inflexible body. In a preferred embodiment, first seal 20 is a flexible band that is reversibly or fixably attached to the external surface 14 or internal surface 12 of flexible device 10. A first seal 20 may be associated with one or both ends of the device. In another preferred embodiment, two adjacent flexible bands 20 and 20′are applied to first end 16 or second end 18 or both to urge each end of flexible device 10 into contact with the skin or body. First seal 20 is “adjacent”to first seal 20′where it is positioned less than halfway between the centerline of the device and one end of the device. Preferably, the adjacent first seals 20 and 20′ are within one quartile of the length of the device and closer to the end of the device than to the centerline of the device. In a more preferred embodiment, the two adjacent first seals 20 and 20″ are within an area equal to one tenth the length of the device and closer to one end of the device than to the centerline of the device.

In another alternate embodiment of this invention, sealing device 10 is reversibly sealed along one axis with a second seal 22 to form a sheet when unsealed and to form a cylinder when sealed. When sealing device 10 includes a second seal 22, sealing device 10 may be applied to a as a sheet and then fitted to the limb by activating second seal 22. For instance, second seal 22 can be a reversible seal in the form of a zipper, or second seal 22 can be an irreversible seal such a an adhesive associated with one or both surfaces being sealed. To apply a sealing device 10 with a second seal 22 to a limb, the second seal is unengaged to form a sheet and the sheet is then placed upon and around a limb or appendage, and second seal 22 zipper is closed or sealed. In this alternative embodiment, sealing device 10 does not need to be pulled over a hand, for instance, to apply the device to a forearm. The device may simply purchased open or it is opened using a reversible second seal such as a zipper, placed around a forearm for example, and then the zipper is closed to form sealing device 10. This embodiment would be useful where, for example, the hand attached to the forearm is broken or otherwise injured and in a cast. It should be understood that second seal 22 may be any suitable mechanism for releasing and resealing the device. Non-limiting examples of such useful seals include zippers, adhesives (i.e., an activatable adhesive or Velcro™), or a plastic-based mechanism such as is used to seal refrigerator bags (i.e., ZipLock™) and so forth.

In another alternate embodiment, the device comprises multiple tubular extensions attached to a central portion, where tubular extensions project from the central portion. This embodiment is similar in structure to a glove or a sock, where the device may be applied to protect a hand, foot, or similar appendage. The fingers or toes of the hand or foot, respectively, may protrude from the tubular extensions of the device, while the palm or sole of the foot is protected by the device.

In still another alternate embodiment, the device may comprise a central portion with one or more holes at the edge through which one or more fingers or toes may protrude. These embodiments of the device may also comprise any of the sealing mechanisms described above.

The invention also provides methods for manufacturing the device comprising molding from a starting material a flexible, tubular structure, and fixably attaching to said tubular structure a first seal to one or both ends of sealing device 10. First seal 20 may be any of those described herein. Sealing device 10 may be constructed of any suitable material provided the material provides the necessary hardness to effectuate the invention while yielding sufficient elasticity. The material is preferably impervious to contaminants such as water, dirt, dust or infectious agents. In one embodiment, a blow molded polyethylene tube may be manufactured and have fixably attached to it a natural rubber band as the sealing mechanism. Blow molded tubing is available in different diameters and thicknesses and can be melt sealed to capture the rubber band or to completely seal an end. One of skill in the art would recognize the many suitable materials for constructing the device.

It will be understood that the embodiment described herein are merely exemplary and that a person skilled in the art may make many variations and modifications without departing from the spirit and scope of the invention. All such variations and modifications are intended to be included within the scope of the invention as defined in the appended claims.

What is claimed is:

1. A skin shielding device comprising a flexible membrane forming a cylinder having an internal surface, an external surface, a first end, and a second end wherein the external surface is impermeable to at least one contaminant, said membrane including at least one first seal.

2. The skin shielding device of claim 1 wherein the membrane is air permeable.

3. The skin shielding device of claim 1 wherein the first seal is a flexible band that is attached to an end of the shielding device selected from the first end and the second end.

4. The skin shielding device of claim 3 wherein the first seal is attached to the shielding device internal surface.

5. The skin shielding device of claim 3 wherein the first seal is attached to the shielding device external surface.

6. The skin shielding device of claim 1 including adjacent first seals.

7. The skin shielding device of claim 1 including a second seal.

8. The skin shielding device of claim 7 wherein the second seal is selected from the group consisting of a zipper, an adhesive, and a zip lock mechanism.

9. The skin shielding device of claim 1 comprising multiple tubular structures a central portion, said tubular structures projecting from said central portion.

10. The skin shielding device of claim 9 wherein said device is suitable for protection of a hand comprising insertion of one or more fingers into the tubular structures projecting from said central portion of the device.

11. The skin shielding device of claim 7 wherein said device is suitable for protection of a foot comprising insertion of one or more toes into the tubular structures projecting from said central portion of the device.

12. A method for applying a skin shielding device comprising a flexible membrane forming a cylinder having an internal surface, an external surface, a first end, and a second end wherein the external surface is impermeable to at least one contaminant, said membrane including at least one first seal to an appendage by the steps comprising:
a. placing an end of the appendage into the first end of the skin shielding device; and
b. passing the end of the appendage through the skin shielding device until a portion of the appendage passes out of the second end of the skin shielding device.

13. The method of claim 12 wherein the appendage is selected from the group consisting of an arm, a leg, a hand, a foot, a finger, and toe, a torso, and a head.

14. The method of claim 12 wherein the shielding device covers an IV shunt on the appendage.

15. The method of claim 12 wherein the shielding device covers a wound on the appendage.
